(19) United States
(12) Patent Application Publication
(10) Pub. No.: US 2026/0215761 A1
(43) Pub. Date: Jul. 30, 2026

(54) MODULAR COMPACT ULTRASOUND IMAGING SYSTEM

(71) Applicant: GE Precision Healthcare LLC, Waukesha, WI (US)

(72) Inventors: Tali Gordon Ben-Ami, Zichron Yaakov (IL); Lionel Wodecki, Maisons Alfort (FR); Fenggui Huang, Wuxi (CN)

(73) Assignee: GE Precision Healthcare LLC, Waukesha, WI (US)

(21) Appl. No.: 18/831,435

(22) Filed: Jan. 24, 2025

(51) Int. Cl. A61B 8/00

(52) U.S. Cl. CPC A61B 8/469; A61B 8/4411; A61B 8/4433; A61B 8/462; A61B 8/464; A61B 8/465; A61B 8/54

(57) ABSTRACT

A modular compact ultrasound imaging system is disclosed. In one example, an ultrasound imaging system includes a user interface unit, a display unit, and a control unit. The user interface unit includes a user interface configured to receive user inputs from a user. The display unit includes a display screen configured to display a graphical user interface, where the graphical user interface is configured to depict ultrasound images. The control unit includes a processing circuit configured to facilitate operation of the ultrasound imaging system by receiving user input signals from the user interface unit, controlling a probe, receiving ultrasound signals from the probe, and causing data to be displayed via the display unit. The user interface unit, the display unit, and the control unit are each separate components and are configured to selectively couple with at least one other unit in more than one configuration.

### FIELD

Embodiments of the subject matter disclosed herein relate to ultrasound imaging, and more particularly, to a modular compact and mobile ultrasound imaging system.

### BACKGROUND

During a medical imaging scan, a plurality of medical images of a patient are obtained by a technician, such as a sonographer, to measure or detect various aspects of anatomical features present within the medical images. The medical imaging scan may be performed in a variety of scenarios such as diagnostic imaging, interventional procedures, and so on.

### SUMMARY

An embodiment relates to an ultrasound imaging system. The ultrasound imaging system includes a user interface unit having a user interface configured to receive user inputs from a user. The ultrasound imaging system includes a display unit having a display screen configured to display a graphical user interface, the graphical user interface configured to depict ultrasound images. The ultrasound imaging system includes a control unit having a processing circuit configured to facilitate operation of the ultrasound imaging system by receiving user input signals from the user interface unit, controlling a probe, receiving ultrasound signals from the probe, and causing data to be displayed via the display unit. The user interface unit, the display unit, and the control unit are each separate components and are configured to selectively couple with at least one other unit in more than one configuration.

Another embodiment relates to an ultrasound imaging system. The ultrasound imaging system includes a user interface unit having a user interface configured to receive user inputs from a user. The ultrasound imaging system includes a display unit having a display screen configured to display a graphical user interface, the graphical user interface configured to depict ultrasound images. The ultrasound imaging system includes a control unit having a processing circuit configured to facilitate operation of the ultrasound imaging system by receiving user input signals from the user interface unit, controlling a probe, receiving ultrasound signals from the probe, and causing data to be displayed via the display unit. The control unit further includes a hinge component configured to directly couple the control unit with one of the user interface unit or the display unit.

Another embodiment relates to an ultrasound imaging system. The ultrasound imaging system includes a user interface unit having a user interface configured to receive user inputs from a user. The user interface unit includes a first display screen configured to display a first graphical user interface. The ultrasound imaging system includes a display unit having a second display screen configured to display a second graphical user interface, the second graphical user interface configured to depict ultrasound images. The user interface unit and the display unit are each configured to separately couple with a hinge component configured to support one of the user interface unit and the display unit.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of an ultrasound imaging system, according to an example embodiment.

FIG. 2 is a block diagram of the ultrasound imaging system of FIG. 1 in greater detail, according to an example embodiment.

FIG. 3 is an illustration of a control unit used in the ultrasound imaging system of FIG. 1, according to an example embodiment.

FIG. 4 is an illustration of a user interface unit used in the ultrasound imaging system of FIG. 1, according to an example embodiment.

FIG. 5 is an illustration of a display unit used in the ultrasound imaging system of FIG. 1, according to an example embodiment.

FIG. 6 is an illustration of a probe connection unit used in the ultrasound imaging system of FIG. 1, according to an example embodiment.

FIG. 7A is an illustration of the ultrasound imaging system of FIG. 1 in a first configuration, according to an example embodiment.

FIG. 7B is another illustration of the ultrasound imaging system of FIG. 1 in the first configuration, according to an example embodiment.

FIG. 8 is an illustration of the ultrasound imaging system of FIG. 1 in a second configuration, according to an example embodiment.

FIG. 9 is an illustration of the ultrasound imaging system of FIG. 1 in a third configuration, according to an example embodiment.

FIG. 10 is an illustration of the ultrasound imaging system of FIG. 1 in a fourth configuration, according to an example embodiment.

FIG. 11A is an illustration of a hinge component of the ultrasound imaging system of FIG. 1, according to an example embodiment.

FIG. 11B is another illustration of the hinge component of the ultrasound imaging system of FIG. 1, according to an example embodiment.

FIG. 11C is another illustration of the hinge component of the ultrasound imaging system of FIG. 1, according to an example embodiment.

FIG. 11D is another illustration of the hinge component of the ultrasound imaging system of FIG. 1, according to an example embodiment.

FIG. 11E is another illustration of the hinge component of the ultrasound imaging system of FIG. 1, according to an example embodiment.

### DETAILED DESCRIPTION

Referring generally to the figures, systems and methods for a modular compact ultrasound imaging system are disclosed. More specifically, the systems and methods described herein provide for an ultrasound imaging system including a control unit, a display unit, and/or a user interface unit that are configurable in various arrangements depending on a desired implementation of the ultrasound imaging system.

In existing ultrasound imaging systems, such systems use equipment (e.g., interface, operating box, display, etc.) in a fixed configuration to perform the ultrasound. Furthermore, these systems cause considerable ergonomic strain on the users.

The systems and methods described herein, however, provide a technical solution to existing systems by providing a compact and mobile ultrasound imaging system. More specifically, the compact and mobile ultrasound imaging system described herein can be used in a variety of modular configurations, depending on user needs across various use scenarios (e.g., during diagnostic echocardiography and echo-guided interventional procedures). The modular concept described herein therefore improves the ergonomics of ultrasound imaging systems, while improving ultrasound imaging workflow.

The implementations described herein address a technical problem by providing enhanced data integration and analysis capabilities, which deliver a particular technical solution that streamlines and refines generation and transmittal of medical images. For instance, the ultrasound imaging system described herein provides for multiple configurations of the individual components, which allows ultrasound users (e.g., sonographers) to perform an ultrasound imaging procedure using ultrasound equipment in a plurality of configurations without having to substitute imaging equipment depending on a desired configuration. Accordingly, this approach provides a specific technical improvement to various technical problems, including those set forth herein. Furthermore, the ergonomic benefits of the ultrasound imaging system described herein allow users to perform ultrasounds for longer durations of time without significant ergonomic strain, which improves efficiency.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

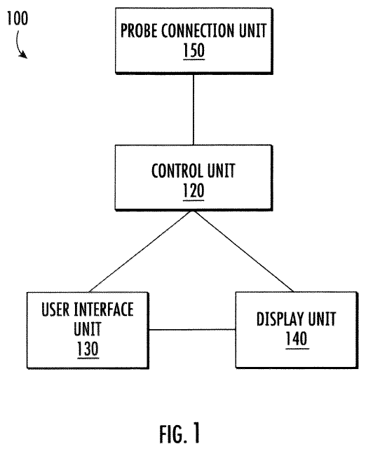

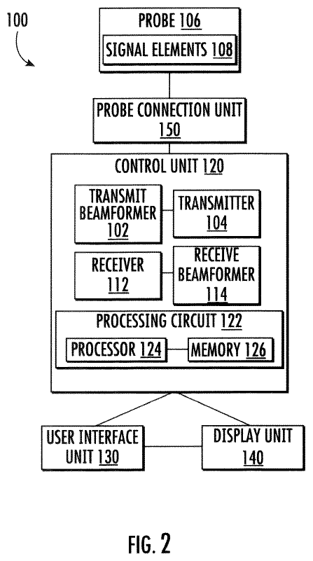

Referring to FIGS. 1 and 2, a block diagram of an ultrasound imaging system 100 is shown. The ultrasound imaging system 100 may be used in a medical environment (e.g., hospitals, clinics, etc.), for example, by a sonographer, technician, or other clinician certified to collect ultrasound data from a patient.

An example of a procedure performed using the ultrasound imaging system 100 may be an echocardiogram. Echocardiograms are performed to detect heart abnormalities in a patient by collecting and processing ultrasound data (e.g., using the ultrasound imaging system 100, as described herein). During an echocardiogram, the sonographer collects the ultrasound data by navigating a probe (e.g., probe 106, as described below) over the patient's chest until a sufficient volume of ultrasound images are collected. The collected images are stored in a central storage device (e.g., memory 126) and analyzed by the sonographer. The sonographer generates a set of measurements from the images (e.g., 50-100 records), and the images and measurements are collectively reviewed by a cardiologist. The cardiologist provides any clinical findings or conclusions in a report submitted to the patient's medical record.

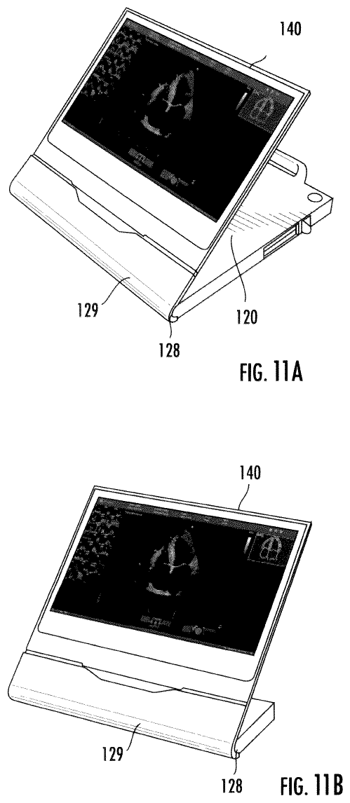

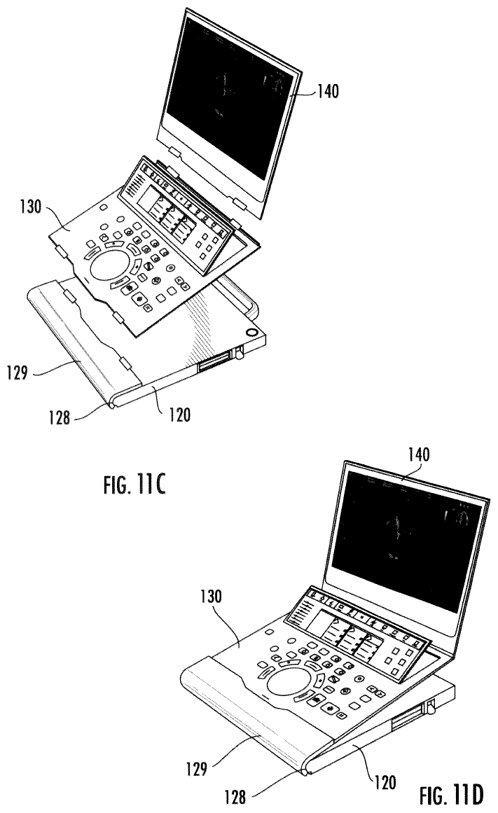

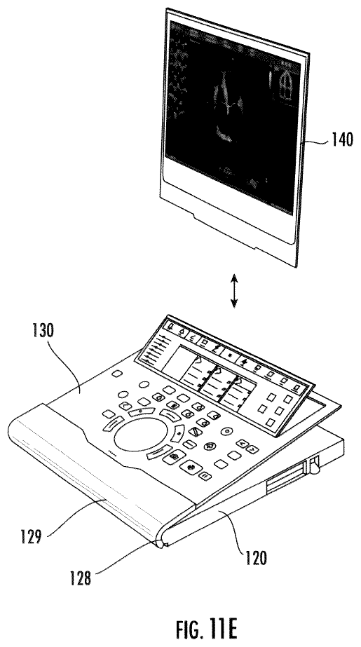

As shown in FIGS. 1 and 2, the ultrasound imaging system 100 includes a probe 106, a control unit 120, a user interface unit 130, a display unit 140, and a probe connection unit 150. As described herein, the control unit 120, the user interface unit 130, the display unit 140, and the probe connection unit 150 may each be separate components of the ultrasound imaging system 100 and may be configured to selectively couple with at least one other unit in more than one configuration. For instance, and as described in greater detail below, FIGS. 11A-11E depict various configurations of the control unit 120 coupled to at least one of the user interface unit 130 or the display unit 140.

Referring to FIG. 2, the ultrasound imaging system 100 is shown to include the probe 106. The probe 106 may be a linear array probe, a curvilinear array probe, a sector probe, or any other type of probe configured to obtain two-dimensional (2D) B-mode data, 2D color flow data, M-mode data, three-dimensional (3D) data, four-dimensional (4D) data, or any other type of ultrasound data. Alternatively or additionally, the probe 106 may be any type of probe configured to obtain 2D B-mode data and data corresponding to another ultrasound mode that detects blood flow velocity in the direction of a vessel axis. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. For example, the anatomy being imaged may be identified as a left atrial appendage (LAA) at a first location of the probe 106. Then, the position sensor may track the movement of the probe 106 relative to the LAA in order to identify successive locations of the probe 106. In some embodiments, the position sensor may transmit position data to be stored within the ultrasound imaging system 100 (e.g., in memory 126).

The probe 106 may include a transducer configured to transmit and receive an ultrasound signal. In some embodiments, as shown in FIG. 2, the probe 106 includes signal elements 108. The signal elements 108 may be arranged in a transducer array, and in some embodiments may be arranged in a one-dimensional (1D) or 2D array. The signal elements 108 emit pulsed ultrasonic signals into a body of a subject (e.g., a patient). For example, during an echocardiogram, a sonographer or other clinician may navigate the probe 106 over a patient's chest so that the signal elements 108 in the probe 106 emit the pulsed ultrasonic signals into the patient's thoracic cavity. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to have an acoustic impedance between a tissue to be imaged and a material of the transducer (e.g., such that the pulsed electronic signals can be back-scattered from the anatomical structures in the body and received as echoes by the signal elements 108), and a damping block configured to absorb ultrasound energy.

As shown in FIG. 2, the control unit 120 may include a transmit beamformer 102, a transmitter 104, a receiver 112, a receive beamformer 114, and a processing circuit 122. The control unit 120 may be configured to facilitate operation of the ultrasound imaging system 100 by, for instance, receiving user input signals from the user interface unit 130, controlling the probe 106, receiving ultrasound signals from the probe 106, and causing data to be displayed via the display unit 140. In some embodiments, as described below with reference to FIG. 8, the control unit 120 may be configured to communicate with the user interface unit 130 and/or the display unit 140 via a network connection (e.g., network 101).

In some embodiments, the control unit 120 includes the transmit beamformer 102 and the transmitter 104. The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 124, as described below) may be configured to perform some or all of the functions associated with the transmit beamformer 102. The transmitter 104 drives the signal elements 108 to emit the pulsed ultrasonic signals into the body of the subject.

The receiver 112 receives the echoes from the probe 106 and converts the echoes into electrical signals. The electrical signals are then passed through the receive beamformer 114, which produces the ultrasound data from the electrical signals. As described above with reference to the transmit beamformer 102, the receive beamformer 114 may be either a hardware beamformer or a software beamformer. In embodiments where the receive beamformer 114 is a hardware beamformer, the receive beamformer 114 may include one or more of a GPU, a microprocessor, a CPU, a DSP, or any other type of processor capable of performing logical operations. The receive beamformer 114 may be configured to perform conventional beamforming techniques as well as techniques such as retrospective transmit beamforming (RTB). Alternatively, in embodiments where the receive beamformer 114 is a software beamformer, a processor (e.g., processor 124, as described below) may be configured to perform some or all of the functions associated with the receive beamformer 114.

Although the transmit beamformer 102, the transmitter 104, the receiver 112, and the receive beamformer 114 are shown in FIG. 2 as being components of the control unit 120 that are distinct from the probe 106, it should be appreciated that in some embodiments, the probe 106 may include electronic circuitry configured to perform the functions of each of the transmit beamformer 102, the transmitter 104, the receiver 112, and/or the receive beamformer 114. That is, all or part of the transmit beamformer 102, the transmitter 104, the receiver 112, and/or the receive beamformer 114 may be situated within the probe 106.

Referring still to FIG. 2, the control unit 120 is shown to include a processing circuit 122. As shown, the processing circuit 122 may include at least one processor 124 and a memory 126. In this way, the processing circuit 122 may be structured or configured to execute or implement the instructions, commands, and/or control processes described herein with respect to the processor 124 and the memory 126. While shown as being separate from the probe 106 in FIG. 2, it will be appreciated that the processing circuit 122 can be part of the probe 106. For example, the processing circuit 122 can be disposed in a handheld housing of the probe 106 (e.g., in the case of the probe 106 being a wireless probe).

The processor 124 may include a CPU, a GPU, a microprocessor, a DSP, a general-purpose single-or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 124 may be shared by multiple circuits (e.g., the circuits of the processor 124 may include or otherwise share the same processor which, in some example embodiments, may execute instructions stored, or otherwise accessed, via different areas of the memory 126). Alternatively or additionally, the processor 124 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 124 may be configured to control the transmit beamformer 102, the transmitter 104, the receiver 112, and the receive beamformer 114. The processor 124 may also be in electronic communication with the probe 106. For purposes of this disclosure, the term “electronic communication” may be defined to include both wired and wireless communications.

In some embodiments, the processor 124 may be configured to control the probe 106 during data acquisition. That is, the processor 124 may control the data acquisition by controlling which of the signal elements 108 are active and by controlling a shape of the beam emitted from the probe 106. Alternatively or additionally, the processor 124 may include a complex demodulator configured to demodulate radio frequency (RF) data obtained by the probe 106 and generate raw data. According to other embodiments, the demodulation of the RF data may be performed by another component of the ultrasound imaging system 100. The processor 124 may perform the processing operations described herein according to a plurality of selectable ultrasound modalities.

Depending on a mode of operation of the ultrasound imaging system 100, the processor 124 may process ultrasound data obtained by the probe 106 according to the mode of operation to generate image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 114) to display space coordinates (e.g., such that the ultrasound data may be displayed as image data). In some embodiments, the mode of operation may allow for video processing by the processor 124 such that a series of images (e.g., processed ultrasound data) may be displayed in real-time while a scanning session or procedure is being performed on a patient. An operator of the ultrasound imaging system 100 (e.g., a sonographer) may switch between various modes in order to obtain a variety of ultrasound data and to perform a complete scan of an anatomical region of interest. For example, the operator may switch between modes using the user interface unit 130 and/or the display unit 140 (e.g., using physical controls, interface inputs representing physical controls, etc.). While the term “image” or “images” are used herein to for the purposes of example, it will be appreciated that such terms cover still images as well as videos, clips, or a series of images for each. For example, in some embodiments, the image or images may include a 1-2 second clip derived from the image data.

The processor 124 performs the processing operations in real-time as the echo signals are received by the receiver 112 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes per second. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. For example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes per second, while other implementations of the ultrasound imaging system 100 may have real-time volume-rates that are slower than 7 volumes per second.

In some embodiments, the ultrasound imaging system 100 may include multiple processors configured to perform the processing operations or functionality described with reference to processor 124. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

The processor 124 may also be in electronic communication with the user interface unit 130 and/or the display unit 140 such that the processor 124 may process ultrasound data obtained by the probe 106 and generate images to display on a display screen (e.g., display screen 142, as described below) of the user interface unit 130 and/or the display unit 140.

As shown in FIG. 2, the processing circuit 122 also includes the memory 126. The memory 126 may be configured to, for example, store processed volumes of data obtained by the ultrasound imaging system 100 (e.g., ultrasound data collected by the probe 106, user inputs received by the user interface unit 130 and/or the display unit 140, etc.). For example, the memory 126 may be a hospital picture archiving and communication system (PACS). The memory 126 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 126 may be or include tangible, non-transient volatile memory or non-volatile memory. The memory 126 may also include database components, object code components, script components, or any other type of information structure for supporting the activities and information structures described in the present application.

In various embodiments, the memory 126 may have varying capacity (e.g., storage space) across embodiments of the ultrasound imaging system 100. For example, the memory 126 may be configured to store at least 60 minutes' worth of ultrasound data. The ultrasound data may be stored in the memory 126 such that the ultrasound data may be retrieved according to an order or time of acquiring the data. That is, the ultrasound data may be stored with a timestamp indicating a time at which the ultrasound data was collected and may be retrieved starting with an oldest time at which the ultrasound data was collected.

Referring to FIGS. 1 and 2, the ultrasound imaging system 100 includes the user interface unit 130. The user interface unit 130 may be used by a sonographer or other clinician to control operation of the ultrasound imaging system 100. For example, the sonographer may use the user interface unit 130 to control the input of patient data, to change a scanning or display parameter, to adjust a segmentation of an anatomical feature depicted in an ultrasound image, and/or to select various other modes, operations, parameters, etc. of the ultrasound imaging system 100. In some embodiments, the user interface unit 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface unit 130 may be an electronic device that was designed and developed for use in a medical environment.

In some embodiments, the user interface unit 130 may communicate with the processor 124 (e.g., the control unit 120) through a wireless protocol (e.g., network 101), such as Wi-Fi, Bluetooth, wireless local area network (WLAN), near-field communication, and so on. Additionally or alternatively, the user interface unit 130 may communicate with the processor 124 through an application programming interface (API).

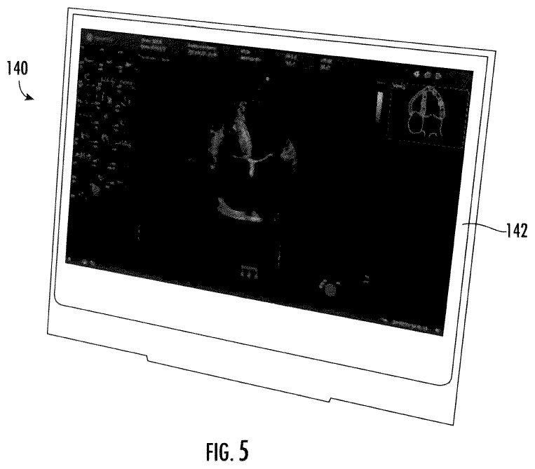

The ultrasound imaging system 100 is also shown to include the display unit 140. The display unit 140 may be configured to display a graphical user interface (GUI) based on an instruction from the memory 126. The GUI may include user interface icons representing commands and instructions relating to the operation of the ultrasound imaging system 100. Additionally or alternatively, and as shown in FIG. 5, the display unit 140 may be configured to display ultrasound images via the GUI based on ultrasound data obtained using the probe 106.

As shown in FIG. 2, the probe connection unit 150 may be configured to connect the control unit 120 to the probe 106. In some instances, the probe connection unit 150 may be configured to receive and house the probe 106 in a stowed position. Furthermore, according to certain implementations, the probe connection unit 150 may be configured to receive and house a plurality of probes 106.

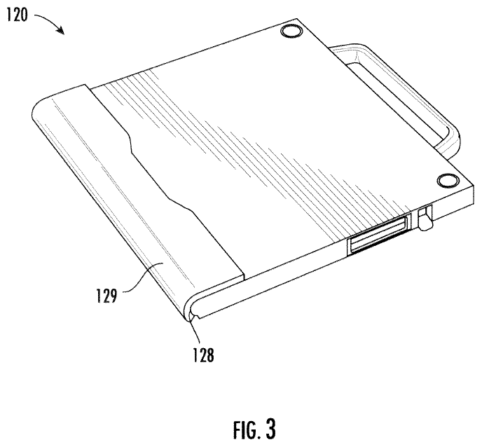

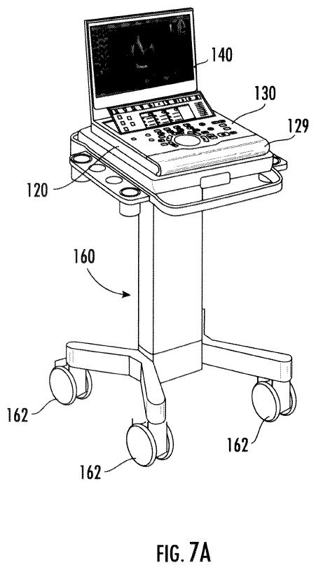

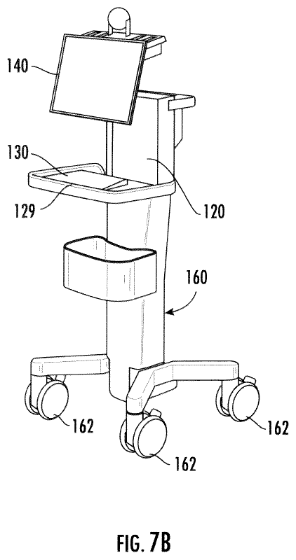

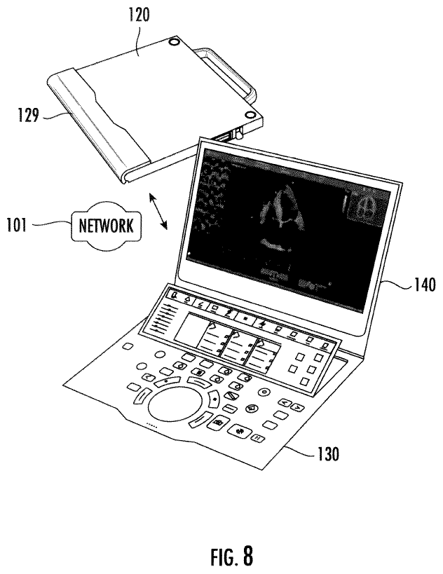

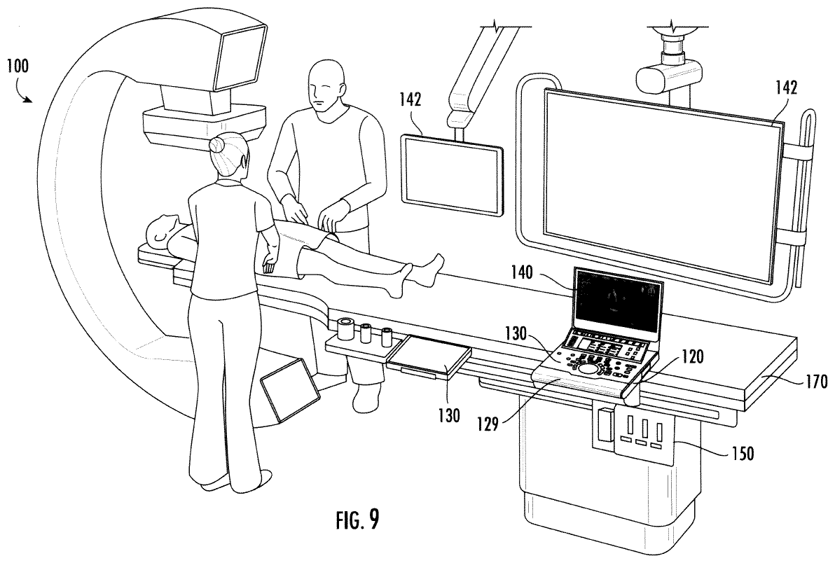

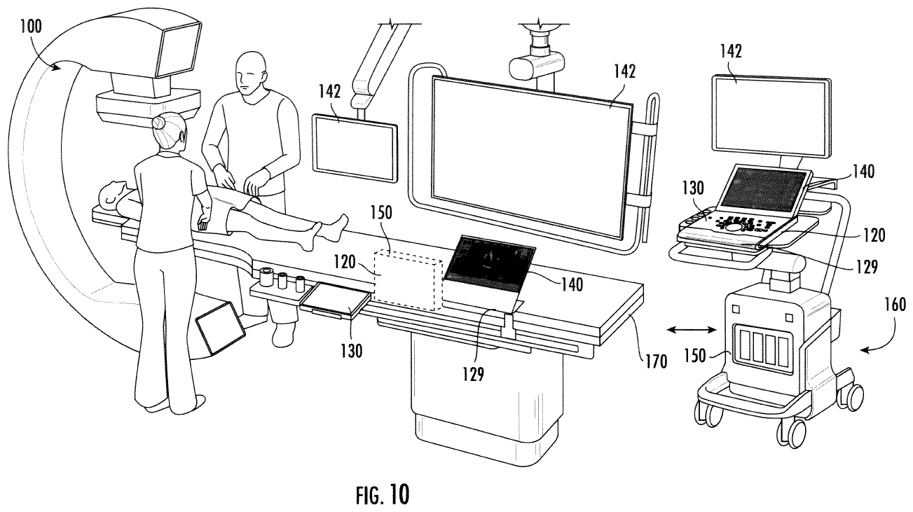

Referring to FIG. 3, the control unit 120 of the ultrasound imaging system 100 is shown in greater detail. The control unit 120 described herein may be used on a cart (e.g., cart 160 as shown in FIGS. 7A-7B and 10), as a laptop (e.g., as shown in FIG. 8), under a bed or table (e.g., table 170 as shown in FIGS. 9-10) in a cardiac catheterization laboratory, and so on. As shown, the control unit 120 may include input/output (I/O) ports such as a probe port (e.g., for the probe 106), an electrocardiogram (ECG) port, among other I/O ports that may be used during an ultrasound imaging procedure (e.g., such as an echocardiogram).

As shown, the control unit 120 may include the hinge component 128. In some embodiments, and as described in greater detail below, the hinge component 128 may be configured to directly couple the user interface unit 130 or the display unit 140 with the control unit 120. Furthermore, the hinge component 128 is adjustable such that the angle of the user interface unit 130 or the display unit 140 with respect to the control unit 120 can be adjusted. For example, the hinge component 128 may be adjustable such that the angle is between 90 degrees and 180 degrees. In some instances, the hinge component 128 further includes a palm rest 129. The palm rest 129 may be configured to enable a user (e.g., a sonographer, clinician, technician, etc.) to rest at least one of a palm, arm, or hand on the palm rest 129 during operation of the ultrasound imaging system 100.

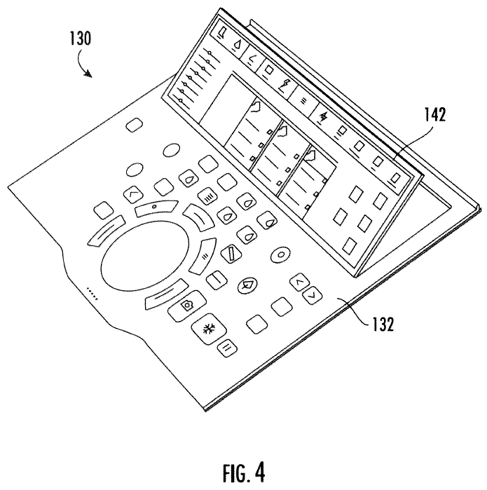

Referring to FIG. 4, the user interface unit 130 of the ultrasound imaging system 100 is shown in greater detail. The user interface unit 130 described herein may be used on a cart (e.g., cart 160 as shown in FIGS. 7A-7B and 10), with a monitor (e.g., display unit 140) as a laptop (e.g., as shown in FIG. 8), on a desk or table (e.g., table 170 as shown in FIGS. 9-10) in a cardiac catheterization laboratory, and so on. As shown in FIG. 4, the user interface unit 130 may include a user interface 132 and a display screen 142. The user interface 132 may be configured to receive user inputs from a user of the ultrasound imaging system 100 (e.g., a sonographer, clinician, technician, etc.). In some embodiments, the user interface 132 may include physical controls such as one or more of buttons, sliders, a rotary knob, a mouse, a keyboard, a trackball, a trackpad, hard keys linked to specific actions, soft keys that may be configured to control different functions, and so on. As shown in FIG. 4, the display screen 142 of the user interface unit 130 is configured to be adjusted with respect to a primary plane (e.g., the user interface 132) of the user interface unit 130 such that an angle of the display screen 142 with respect to the primary plane is adjustable.

Additionally or alternatively, the display screen 142 may be configured to display a GUI including user interface icons representing commands and instructions relating to the operation of the ultrasound imaging system 100. The user interface icons of the GUI may be configured such that the user may select a specific user interface icon in order to initiate a specific function controlled by the GUI. For example, various user interface icons may be used to represent windows, menus, buttons, cursors, scroll bars, and so on. That is, the physical controls of the user interface 132 may be included as individual hardware elements, as user interface icons displayed on the display screen 142, or as a combination of hardware elements and user interface icons.

In some embodiments, the display screen 142 may include a touch-sensitive display device or a touch screen. According to such embodiments, the touch screen may be configured to interact with the GUI displayed by the display screen 142 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user.

Referring to FIG. 5, the display unit 140 of the ultrasound imaging system 100 is shown in greater detail. In some embodiments, the display unit 140 refers to a monitor that may be implemented on a cart (e.g., cart 160 as shown in FIGS. 7A-7B and 10), as a laptop (e.g., as shown in FIG. 8), on a desk or table (e.g., table 170 as shown in FIGS. 9-10) in a cardiac catheterization laboratory, and so on. Additionally or alternatively, the display unit 140 may be coupled to a hinge component (e.g., the hinge component 128 including the palm rest 129, as described above). In such instances, as shown in FIGS. 10-11B, the display unit 140 may be configured as a tablet (e.g., a remote-control standalone tablet).

As shown in FIG. 5, the display unit 140 may include the display screen 142. The display screen 142 may be configured to display a GUI. In some embodiments, the GUI may be configured to present ultrasound image data (e.g., obtained using the probe 106) to a user of the ultrasound imaging system 100 and/or a patient. Additionally or alternatively, the GUI may include user interface icons representing commands and instructions relating to the operation of the ultrasound imaging system 100. The user interface icons of the GUI may be configured such that a user (e.g., the sonographer, clinician, etc.) may select a specific user interface icon in order to initiate a specific function controlled by the GUI. For example, various user interface icons may be used to represent windows, menus, buttons, cursors, scroll bars, and so on. In this way, the display unit 140 may be used by a user in a similar and/or identical manner as the user interface unit 130 to control operation of the ultrasound imaging system 100. As described above with reference to FIG. 4, the display screen 142 may include the touch-sensitive display device or the touch screen.

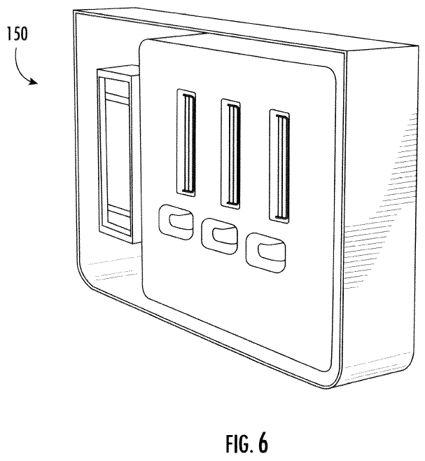

Referring to FIG. 6, the probe connection unit 150 of the ultrasound imaging system 100 is shown in greater detail. In some embodiments, the probe connection unit 150 refers to a multi-probe box that may be implemented on a cart (e.g., cart 160 as shown in FIGS. 7A-7B and 10), on a desk or table (e.g., table 170 as shown in FIGS. 9-10) in a cardiac catheterization laboratory, and so on. More specifically, the probe connection unit 150 may be mounted on the cart 160. Additionally or alternatively, the probe connection unit 150 may be integrated as a part of the cart 160 (e.g., as shown in FIG. 10). As described above, the probe connection unit 150 may be configured to connect the control unit 120 to the probe 106.

Referring to FIGS. 7A-7B, the ultrasound imaging system 100 is shown according to a first configuration. More specifically, the first configuration of the ultrasound imaging system 100 refers to a configuration on a cart 160. As shown in FIGS. 7A-7B, the cart 160 includes a plurality of tractive elements 162 (e.g., wheels) configured to facilitate movement of the cart 160. The cart 160 is shown to further include a center column positioned above a base (e.g., the base coupled to the tractive elements 162). With the configuration shown in FIG. 7A, the control unit 120, the user interface unit 130, and the display unit 140 may be coupled to each other to form a single device (e.g., a laptop). In this way, the single device may be coupled to or otherwise mounted on the cart 160. On the other hand, with the configuration shown in FIG. 7B, each of the control unit 120, the user interface unit 130, and the display unit 140 may be independently coupled to the cart 160. For instance, the display unit 140 may be coupled to an upper portion of the cart 160, the user interface unit 130 may be coupled to a front-facing middle portion of the cart 160, and the control unit 120 may be integrated within an inner portion of the cart 160. In some embodiments, the control unit 120, the user interface unit 130, and/or the display unit 140 may be coupled to each other and/or to the cart 160 via one or more wires within the center column of the cart 160. Furthermore, in some instances, the probe connection unit 150 may be coupled to a rear panel of the cart 160 (e.g., although not visible from the perspective of FIGS. 7A-7B).

With the configuration shown in FIGS. 7A-7B, the user interface unit 130 may be positioned at a hands-height position (e.g., such that a height at which the user interface unit 130 is coupled to the cart 160 approximately matches a height of a user's hands while the user interacts with the user interface unit 130). Furthermore, with the configuration shown in FIGS. 7A-7B, the display unit 140 may be positioned at an eyes-height position (e.g., such that a height at which the display unit 140 is coupled to the cart 160 approximately matches a height of a user's eyes while the user interacts with the display unit 140). More specifically, with the configuration shown in FIG. 7A, the user interface unit 130 and the display unit 140 may be positioned at the hands-height position and the eyes-height position, respectively, using a single adjustment (e.g., to the single device, to the cart 160, etc.). On the other hand, with the configuration shown in FIG. 7B, the position of the user interface unit 130 and the position of the display unit 140 may be adjusted independently from each other. In this way, the configuration shown in FIG. 7B addresses ergonomic strain that may be experienced by a user of existing ultrasound imaging systems by facilitating independent adjustment of a height of the user interface unit 130 (e.g., based on a user-specific hand-height) and a height of the display unit 140 (e.g., based on a user-specific eye-height).

Referring to FIG. 8, the ultrasound imaging system 100 is shown according to a second configuration. More specifically, the second configuration of the ultrasound imaging system 100 refers to a portable computing device (e.g., a laptop) configuration. As shown in FIG. 8, the second configuration includes the control unit 120, the user interface unit 130, and the display unit 140. In some instances, each of the control unit 120, the user interface unit 130, and the display unit 140 may be directly coupled together to form a single device (e.g., as shown in FIG. 9). Additionally or alternatively, the user interface unit 130 and the display unit 140 may be directly coupled together to form the portable computing device. In such embodiments, as shown in FIG. 8, the portable computing device (e.g., the user interface unit 130 and the display unit 140) may be connected to the control unit 120 via a network 101. The network 101 may include one or more of the Internet, cellular network, Wi-Fi, Wi-max, a proprietary network, or any other type of wired or wireless network of a combination of wired or wireless networks. The network 101 may facilitate communication between the respective components of the ultrasound imaging system 100 (e.g., the control unit 120, the user interface unit 130, the display unit 140, etc.), as described herein. In some instances, the second configuration shown in FIG. 8 may include a second palm rest (e.g., palm rest 129) without a hinge component (e.g., hinge component 128). In this way, the second palm rest may interface with the user interface unit 130 independent from the control unit 120 (e.g., when the user interface unit 130 is not directly coupled to the control unit 120).

Referring to FIGS. 9 and 10, the ultrasound imaging system 100 being implemented in a medical setting such as a cardiac catheterization laboratory is shown. In such implementations, the ultrasound imaging system 100 (e.g., the control unit 120, the user interface unit 130, the display unit 140, the probe connection unit 150) may be coupled to a stationary object such as a table (e.g., table 170), a bed, a stand, a mounting arm (e.g., mounted to the bed, the table, the stand, a boom, etc.) and so on. For instance, as shown in FIGS. 9 and 10, the ultrasound imaging system 100 is coupled to the table 170 in the cardiac catheterization laboratory. In some instances, as shown in in FIGS. 9 and 10, such a configuration of the ultrasound imaging system 100 (e.g., in the cardiac catheterization laboratory) may include additional display screens 142 (e.g., in addition to the display screen 142 included in the display unit 140 and/or the display screen 142 included in the user interface unit 130). In this way, any of the display screens 142 may be used to present ultrasound image data to a user (e.g., sonographer, clinician, technician, etc.) and/or patient.

As shown in FIG. 9, the ultrasound imaging system 100 (e.g., the control unit 120, the user interface unit 130, the display unit 140) may be configured as a computing device (e.g., a laptop, a tablet, etc.) coupled to the table 170. That is, one or more of the control unit 120, the user interface unit 130, and the display unit 140 are configured to couple to the table 170. In some instances, the control unit 120 may be coupled to an underside of the table 170. Additionally or alternatively, the control unit 120 may be coupled to a table rail coupled to a side of the table 170. Furthermore, as shown in FIG. 9, the probe connection unit 150 may be coupled to the table 170 to facilitate implementation and use of the ultrasound imaging system 100 in the cardiac catheterization laboratory. In some embodiments, the control unit 120 and the probe connection unit 150 may be connected via a wired connection. The ultrasound imaging system 100 may also include more than one user interface unit 130, as shown in FIG. 9, such that user(s) may control operation of the ultrasound imaging system 100 via the more than one user interface unit 130.

Referring to FIG. 10, the ultrasound imaging system 100 (e.g., the control unit 120, the user interface unit 130, the display unit 140) may be configured as a remote (e.g., hand-held, wireless, etc.) computing device (e.g., a tablet). In this way, as shown in FIG. 10, the display unit 140 may be configured as a removable device for use on the table 170, the cart 160, and so on. In such instances, the control unit 120 and/or the probe connection unit 150 may be coupled to the underside of the table 170, as shown in FIG. 10. Additionally or alternatively, as shown in FIG. 10, the control unit 120 and/or the probe connection unit 150 may be coupled to the cart 160. In this way, the display unit 140 may be configured to facilitate a remote control of the ultrasound imaging system 100 via a wireless connection with the control unit 120. In some embodiments, as described above, the display unit 140 may be configured to perform the operations of the user interface unit 130 such that the user may control operations of the ultrasound imaging system 100 via the display unit 140 instead of or in addition to the user interface unit 130. For example, as shown in FIG. 10, the ultrasound imaging system 100 may include a user interface unit 130 coupled to the table 170 such that a user at the table 170 may control operation of the ultrasound imaging system 100 via the user interface unit 130, while a remote user (e.g., standing at the cart 160 and/or otherwise remote from the table 170) may control operation of the ultrasound imaging system 100 via the display unit 140.

Referring to FIGS. 11A-11E, various configurations of the ultrasound imaging system 100 are shown. More specifically, each of the various configurations illustrate the hinge component 128 and the palm rest 129 of the ultrasound imaging system 100, as described herein. For instance, as shown in FIG. 11A, the hinge component 128 and the palm rest 129 may be coupled to the control unit 120. In this way, the display unit 140 may be coupled to the control unit 120 via the hinge component 128. As another example, and as shown in FIG. 11B, the hinge component 128 and the palm rest 129 may be coupled to the display unit 140 such that the ultrasound imaging system 100 is configured to include a portable computing device (e.g., a tablet) including the palm rest 129. Additionally or alternatively, as shown in FIGS. 11C and 11D, the hinge component 128 may be coupled to the user interface unit 130. In such instances, the user interface unit 130 may be coupled to the control unit 120 via the hinge component 128. Furthermore, the display unit 140 may be coupled to the user interface unit 130. In some instances, the display unit 140 may directly coupled to the user interface unit 130, as shown in FIG. 11D, thereby configuring the ultrasound imaging system 100 as a portable computing device (e.g., a laptop). Additionally or alternatively, the display unit 140 may be wirelessly coupled to the user interface unit 130 and the control unit 120, as shown in FIG. 11E.

As described herein, the hinge component 128 is configured to support the user interface unit 130 or the display unit 140 on a surface (e.g., on the cart 160, on the table 170, on the control unit 120, etc.). Furthermore, the hinge component 128 is adjustable such that the angle of the user interface unit 130 or the display unit 140 with respect to the surface is adjustable. In this way, the hinge component 128 allows the user to choose a preferred operating angle, which minimizes ergonomic strain during prolonged use of the user interface unit 130 and/or display unit 140. Further, the hinge component 128 allows users to elevate a height of the display unit 140 such that the display unit 140 is configured at an optimized ergonomic viewing angle. As shown, the palm rest 129 includes a round-shaped edge that spans across a total width of the hinge component 128, thus facilitating a relaxed user interaction with the interface unit 130 and/or display unit 140 from a front and/or a diagonal position.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

What is claimed is:

1. An ultrasound imaging system comprising:
a user interface unit comprising a user interface configured to receive user inputs from a user;
a display unit comprising a display screen configured to display a graphical user interface, wherein the graphical user interface is configured to depict ultrasound images; and
a control unit comprising a processing circuit configured to facilitate operation of the ultrasound imaging system by receiving user input signals from the user interface unit, controlling a probe, receiving ultrasound signals from the probe, and causing data to be displayed via the display unit;
wherein the user interface unit, the display unit, and the control unit are each separate components and are configured to selectively couple with at least one other unit in more than one configuration.

2. The ultrasound imaging system of claim 1, further comprising a probe connection unit configured to receive and house the probe in a stowed position.

3. The ultrasound imaging system of claim 2, wherein the probe connection unit is configured to receive and house a plurality of probes.

4. The ultrasound imaging system of claim 1, wherein one or more of the user interface unit, the display unit, and the control unit are configured to couple with a cart configured to be moved via a plurality of wheels.

5. The ultrasound imaging system of claim 1, wherein one or more of the user interface unit, the display unit, and the control unit are configured to couple with a stationary object, wherein the stationary object comprises at least one of a stand, table, or bed.

6. The ultrasound imaging system of claim 1, wherein the control unit further comprises a hinge component, and the user interface unit or the display unit being directly coupled with the control unit by the hinge component.

7. The ultrasound imaging system of claim 6, wherein the hinge component is adjustable such that an angle of the user interface unit or the display unit with respect to the control unit can be adjusted.

8. The ultrasound imaging system of claim 7, wherein the angle is between 90 degrees and 180 degrees.

9. The ultrasound imaging system of claim 6, wherein the hinge component comprises a palm rest configured to enable the user to rest at least one of a palm, arm, or hand on the palm rest during the operation of the ultrasound imaging system.

10. An ultrasound imaging system comprising:
a user interface unit comprising a user interface configured to receive user inputs from a user;
a display unit comprising a display screen configured to display a graphical user interface, wherein the graphical user interface is configured to depict ultrasound images; and
a control unit comprising a processing circuit configured to facilitate operation of the ultrasound imaging system by receiving user input signals from the user interface unit, controlling a probe, receiving ultrasound signals from the probe, and causing data to be displayed via the display unit;
wherein the control unit further comprises a hinge component configured to directly couple the control unit with one of the user interface unit or the display unit.

11. The ultrasound imaging system of claim 10, wherein the hinge component is adjustable such that an angle of the user interface unit or the display unit with respect to the control unit is adjustable.

12. The ultrasound imaging system of claim 11, wherein the angle is between 90 degrees and 180 degrees.

13. The ultrasound imaging system of claim 11, wherein the user interface unit is directly coupled with the control unit, and the display unit is directly coupled with the user interface unit.

14. The ultrasound imaging system of claim 13, wherein the display unit is configured to be adjusted with respect to the user interface unit such that an angle of the display unit with respect to the user interface unit is adjustable.

15. The ultrasound imaging system of claim 10, wherein the display screen is a first display screen, and the user interface unit further comprising a second display screen.

16. The ultrasound imaging system of claim 15, wherein the second display screen is configured to be adjusted with respect to a primary plane of the user interface unit such that an angle of the second display screen with respect to the primary plane is adjustable.

17. An ultrasound imaging system comprising:
a user interface unit comprising a user interface configured to receive user inputs from a user, the user interface unit comprising a first display screen configured to display a first graphical user interface; and
a display unit comprising a second display screen configured to display a second graphical user interface, wherein the graphical user interface is configured to depict ultrasound images;
wherein the user interface unit and the display unit are each configured to separately couple with a hinge component configured to support one of the user interface unit and the display unit.

18. The ultrasound imaging system of claim 17, wherein the hinge component is configured to support the user interface unit or the display unit on a surface, and the hinge component is adjustable such that an angle of the user interface unit or the display unit with respect to the surface is adjustable.

19. The ultrasound imaging system of claim 17, wherein the hinge component is part of a control unit comprising a processing circuit configured to facilitate operation of the ultrasound imaging system by receiving user input signals from the user interface unit, controlling a probe, receiving ultrasound signals from the probe, and causing data to be displayed via the first display screen and the second display screen.

20. The ultrasound imaging system of claim 19, further comprising a probe connection unit configured to couple with the control unit, the probe connection unit configured to receive and house the probe.
